(19) United States
(12) Patent Application Publication
(10) Pub. No.: US 2024/0393322 A1
(43) Pub. Date: Nov. 28, 2024

(54) METHODS FOR DETECTING HEAVY METALS IN BIOLOGICAL SAMPLES

(71) Applicant: The Board of Trustees of the Leland Stanford Junior University, Stanford, CA (US)

(72) Inventors: Mary PRUNICKI, Stanford, CA (US); Kari C. NADEAU, Stanford, CA (US); Eric M. SMITH, Stanford, CA (US)

(73) Assignee: The Board of Trustees of the Leland Stanford Junior University, Stanford, CA (US)

(21) Appl. No.: 18/692,943

(22) PCT Filed: Sep. 22, 2022
(86) PCT No.: PCT/US2022/076864

§ 371 Date: Mar. 18, 2024

Related U.S. Application Data
(60) Provisional application No. 63/248,110, filed on Sep. 24, 2021.

(51) Int. Cl. G01N 33/53; G01N 33/569

(52) U.S. Cl. CPC G01N 33/5308; G01N 33/56972

(57) ABSTRACT

The present disclosure provides methods for detecting heavy metals in tissues and cells while identifying cell types at the single-cell level with intracellular or cell surface heavy metal content. The cell type can be identified by contacting the cell with an antibody that binds a biomarker conjugated to a heavy metal that is different from the heavy metal detected in or on the surface of the cell. The methods include detecting heavy metals using mass cytometry.

### CROSS-REFERENCE TO RELATED PATENT APPLICATIONS

This application claims the benefit of priority to U.S. Provisional Application No. 63/248,110, filed Sep. 24, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under contract NIEHS R01ES020926 awarded by the National Institutes of Health. The Government has certain rights in the invention.

### BACKGROUND

Heavy metal (HM) exposure has been linked to immune system dysregulation (1) and the mode of exposure may include dermal contact and absorption, ingestion of contaminated water and food, and inhalation of pollutants (2). Importantly, HMs are often a component of particulate matter from combustion sources, such as wildfire smoke and fossil fuels (2,3). Given the increase in exposures associated with pollution and/or climate change, which potentially may increase HM exposure in air, water, and food sources, the need for new targeted methods of detection is paramount.

A growing body of literature demonstrates detrimental health impacts caused by HM exposure in early childhood, which is a critical time for immune development. HMs, such as lead and cadmium, accumulate in the body and are stored in various tissues, indicating rapid storage in children (4). Exposure to HM may even occur before birth, as mercury, lead, and cadmium are known to cross the placenta and accumulate in fetal tissue (5), potentially causing immediate and delayed effects. For example, at birth, Wai et al, reported that prenatal maternal cadmium exposure was associated with low birth weights within a cohort of pregnant women in Myanmar (6). In later childhood years, an association between early-life low-level cadmium exposure and lower IQ scores in a 5-year-old cohort was reported (7), along with elevated cadmium levels in pediatric cancer patients in Egypt (8). Similar neurological findings have been demonstrated in children after reported exposure to other heavy metals. Lead and mercury were reported to be elevated in a cohort of Egyptian autistic children when compared to controls (9), and blood lead levels in 7-8 year old children was correlated with greater levels of autistic behavior at age 11-12 in a Korean cohort (10). Mechanistically, placental glucocorticoid receptor (NR3CC1) methylation is linked with neurobehavioral risk. For example, Appleton et al., reported increased NR3CC1 methylation with greater levels of arsenic, cadmium, lead, manganese, and mercury exposure (11).

Concentrations of heavy metals in biological samples are typically detected with inductively coupled plasma-mass spectrometry (ICP-MS) (12,13). To date, however, no method exists to identify immune cell types at the single-cell level and simultaneously quantify cell surface or intracellular HM content. The instant disclosure provides a solution to this problem.

### BRIEF SUMMARY

The present disclosure provides methods for detecting heavy metals in a biological sample. The methods provide the advantage of identifying cell types at the single-cell level combined with determining intracellular or cell surface heavy metal content in the same assay. The method also provides the advantage of detecting more than one heavy metal in a biological sample in the same assay, for example, the method can detect 1, 2, 3, 4, 5, 6, 7, 8, 9, 10, 11, 12, 13, or more heavy metals from one or more biological samples in the same assay.

In one aspect, the disclose provides a method for detecting an amount of one or more heavy metals in a biological sample, the method comprising:
(a) contacting the biological sample with one or more antibodies conjugated to a heavy metal; and
(b) detecting both the amount of heavy metals conjugated to the one or more antibodies and the amount of the one or more heavy metals in the biological sample, wherein the one or more heavy metals in the biological sample are not conjugated to an antibody.

In some embodiments, the heavy metal conjugated to the one or more antibodies is different than the one or more heavy metals detected in the biological sample.

In some embodiments, the one or more heavy metals are detected by mass cytometry, cytometry by time-of-flight (CyTOF), inductively coupled plasma-mass spectrometry (ICP-MS), inductively coupled plasma time-of-flight mass spectrometry (ICP-TOF-MS) or epigenetic landscape profiling using cytometry by Time-Of-Flight (EpiTOF).

In some embodiments, the one or more heavy metals comprise one or more isotopes in the range of 75-209 atomic mass units (AMU). In some embodiments, the one or more heavy metals are selected from the group consisting of arsenic, selenium, bromide, rhodium, cadmium, antimony, barium, tantalum, tungsten, osmium, platinum, mercury, lead, and combinations thereof. In some embodiments, the one or more isotopes are selected from the group consisting of 75As, 78Se, 80Se, 79Br, 81Br, 103Rh, 106Cd, 108Cd, 110Cd, 111Cd, 112Cd, 114Cd, 116Cd, 121Sb, 123Sb, 138Ba, 180Ta, 181Ta, 182W, 184W, 186W, 187Os, 1880s, 1890s, 194 Pt, 195 Pt, 196Hg, 198Hg, 199Hg, 200Hg, 201Hg, 202Hg, 204Pb, 206Pb, 207Pb, 208Pb, and combinations thereof.

In some embodiments, the one or more antibodies are conjugated to a heavy metal isotope in the range of 75-209 atomic mass units (AMU). In some embodiments, the antibody is conjugated to a heavy metal isotope selected from the group consisting of 89Y, 141Pr, 142Nd, 149Sm, 152Sm, 175Lu, 165Ho, 169Tm, 146Nd, 163Dy, 171Yb, 158Gd, 176Yb, 173Yb, 151Eu, 150Nd, 162Dy, 147Sm, 159Tb, 155Gd, 154Sm, 168Er, 160Gd, 161Dy, 209Bi, 144Nd, 170Er, 143Nd, 174Yb, 113 In, 164Dy, 153Eu, 156Gd, 167Er, 172Yb, and combinations thereof.

In some embodiments, the antibody specifically binds to CD45, CD3, CD19, CD56, CD66, CD66b, CD14, CD16, CD32, CD64, CD33, CD68, CD115, CD1c, CD141, CD123, FceR1a, CD11c, CD303, PD-11, CD36, CD163, CD206, CLEC4D, CD370, CD47, CD11b, CD54, CD62L, HLA-DR, CD86, CCR1, CCR2, CCR5, CCR7, CX3CR1, or combinations thereof.

In some embodiments, the biological sample comprises a cell or tissue. In some embodiments, the cell is selected from the group consisting of an immune cell, a peripheral blood mononuclear cell (PBMC), a leukocyte, a dendritic cell, and combinations thereof. In some embodiments, the leukocyte is selected from the group consisting of a B cell, a T cell, a myeloid cell, a granulocyte, a natural killer cell, and combinations thereof. In some embodiments, the T cell is selected from the group consisting of a helper T cell, a cytotoxic T cell, a memory T cell, a regulatory T cell (Treg), a natural killer T cell, and combinations thereof. In some embodiments, the helper T cell is a subtype selected from the group consisting of Th1, Th2, Th9, Th17, Tfh, and combinations thereof.

In some embodiments, the cell is a single cell. In some embodiments, the cell is a living cell. In some embodiments, the cell is a dead cell, a cryopreserved cell, a fixed cell, or is obtained from cadaveric tissue. In some embodiments, the cell or tissue is selected from an animal, a mammal, a human, a plant, a single cell eukaryotic organism, or a bacterium.

In some embodiments, the heavy metal detected is on the cell surface. In some embodiments, the heavy metal detected is intracellular.

In some embodiments, the biological sample is obtained from a subject exposed to heavy metals. In some embodiments, the biological sample is obtained from a subject exposed to air pollution. In some embodiments, the biological sample is obtained from a subject exposed to smoke, such as smoke from combustion of fossil fuels, or from wild-fire or forest-fire smoke.

### BRIEF DESCRIPTION OF THE DRAWINGS

FIG. 1: Dead cell comparisons in smoke-exposed firefighters versus controls. Gating strategy for live versus dead versus total cells. Cells were selected based on 193-Ir and 195-Ir staining, which discriminates live from dead cells. Plots were made for live versus dead versus total cells (FIG. 1A). Next, levels of heavy metal isotopes for the dead cell population in smoke-exposed firefighters (n=20) were compared to age and sex-matched controls (n=22). Examples are shown in FIG. 1B for Tantalum isotope 181 and FIG. 1C for Mercury isotope 196. Independent T-tests were performed, and p values reported. The y axis is the percent frequency of the heavy metal isotope and the x axis represents the 2 cohorts.

FIG. 2: Gating strategy for heavy metal positive live cell leukocytes. Intact cells were first selected based on 193-Ir and 195-Ir staining. Doublets and 176Lu and 140Ce beads were removed. Live cells were gated off of 115 IN staining and CD45+ leukocytes were then selected. Myeloid cells were separated from B and T cells through exclusion of CD3+ and CD19+ cells.

FIG. 3: Counts and percentages of CD45+ cells live cells with detectable heavy metals. Heavy metals were gated in cells with the following isotopes 78_80 Selenium, 79_81 Bromide, 112_116 Cadmium, 121_123 Antimony, 180_181 Tantalum, 182_184 Tungsten, 196_198 Mercury, and 207_208 Lead. Counts were determined for each samples HM+ cells (FIG. 3A), followed by conversion to percentage of metal isotope cells per all CD45+ cells (FIG. 3B). A log scale Y axis was used for each graph.

FIG. 4. Percentages of live cells for lead positive cells versus leukocyte phenotypes with detectable 208Lead. Gating of lead positive cells generated distinct populations of CD3−/CD19+ B cells, CD3+/CD19− T cells, and CD3−/CD19− Myeloid and NK Cells (FIG. 4A). Further gating identified CD66−/CD56−/CD14+ or CD16+ monocytes. CD66−/CD56−/CD14−/CD16− dendritic cells, CD56+/CD66− NK cells, and CD56−/CD66+ neutrophils (FIG. 4B). Amount of each 208Pb+ phenotype as percentage of total lead cells.

### DETAILED DESCRIPTION

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% (e.g., plus or minus 1%, 2%, 3%, 4%, 5%, 6%, 7%, 8%, 9% or 10%) of the provided value, or rounded to the nearest significant figure, in all cases inclusive of the provided value. Where ranges are provided, they are inclusive of the boundary values.

The term “heavy metal” (“HM”) refers to an element or isotope that is detected by a method of the present disclosure. It will be understood that there is no generally accepted definition of the term heavy metal. For the purposes of this disclosure, the term heavy metal includes, but is not limited to, arsenic, selenium, bromide, rhodium, cadmium, antimony, barium, tantalum, tungsten, osmium, platinum, mercury, and lead. The term also includes stable isotopes of arsenic, selenium, bromide, rhodium, cadmium, antimony, barium, tantalum, tungsten, osmium, platinum, mercury, and lead. The term also refers to heavy metals that are typically found in environmental pollutants, such as contaminated water and food, and in particulate matter from combustion sources, such as fossil fuels and wildfire smoke, and also includes metals that are considered toxic to humans, animals or plants. The term also includes a metallic element with a density greater than 5.

### Methods

The present disclosure provides methods for detecting heavy metals in a biological sample. The methods provide the advantage of identifying cell types at the single-cell level combined with determining intracellular or cell surface heavy metal content in the same assay. In one aspect, the methods comprise labeling the biological sample with an antibody conjugated to a heavy metal, and detecting the amount of heavy metals in the biological sample, where the heavy metal conjugated to the antibody is different than the heavy metal detected in the biological sample. In some embodiments, the method comprises (a) contacting the biological sample with one or more antibodies conjugated to a heavy metal; and (b) detecting both the amount of heavy metals conjugated to the one or more antibodies and the amount of the one or more heavy metals in the biological sample, wherein the one or more heavy metals in the biological sample are not conjugated to an antibody. The method also provides the advantage of detecting more than one heavy metal in a biological sample in the same assay, for example, the method can detect 1, 2, 3, 4, 5, 6, 7, 8, 9, 10, 11, 12, 13, or more heavy metals from one or more biological samples in the same assay. In some embodiments, the representative heavy metals detected by the method include arsenic, selenium, bromide, rhodium, cadmium, antimony, barium, tantalum, tungsten, osmium, platinum, mercury, and lead.

In some embodiments, the method detects one or more isotopes of a heavy metal in a biological sample, for example, the method can detect 1, 2, 3, 4, 5, 6, 7, 8, 9, 10, 11, 12, 13, 14, 15, 16, 17, 18, 19, 20, 25, 30, or more heavy metal isotopes in a biological sample. In some embodiments, the heavy metal isotope is a stable isotope. In some embodiments, the heavy metal isotope detected has an atomic mass unit (AMU) in the range of 75-209. The method also provides the advantage of detecting more than one isotope of a heavy metal in a biological sample in the same assay. Thus, the method can detect more than one, or any combination or sub-combination, of heavy metal isotopes in the same assay. Representative isotopes detected by the method include ⁷⁵As, ⁷⁸Se, ⁸⁰Se, ⁷⁹Br, ⁸¹Br, ¹⁰³Rh, ¹⁰⁶Cd, ¹⁰⁸Cd, ¹⁰Cd, ¹¹¹Cd, ¹¹²Cd, ¹¹⁴Cd, ¹⁶Cd, ¹²¹Sb, ¹²³Sb, ¹³⁸Ba, ¹⁸⁰Ta, ¹⁸Ta, ¹⁸²W, ¹⁸⁴W, ¹⁸⁶W, ¹⁸⁷Os, ¹⁸⁸Os, ¹⁸⁹Os, ¹⁹⁴Pt, ¹⁹⁵Pt, ¹⁹⁶Hg, ¹⁹⁸Hg, ¹⁹⁹Hg, ²⁰⁰Hg, ²⁰¹Hg, ²⁰²Hg, ²⁰⁴Pb, ²⁰⁶Pb, ²⁰⁷Pb, and ²⁰⁸Pb, or a combination or sub-combination thereof.

The method also provides the advantage of detecting multiple cell types from a biological sample in the same assay. In some embodiments, the cell type is detected by contacting the biological sample with an antibody conjugated to a heavy metal. In some embodiments, the antibody is conjugated to a heavy metal isotope in the range of 75-209 atomic mass units (AMU). In some embodiments, the heavy metal conjugated to the antibody is different than the heavy metal detected in the biological sample. For example, the antibody can be conjugated to Yttrium (Y), Praseodymium (Pr), Neodymium (Nd), Samarium (Sm), Lutetium (Lu), Holmium (Ho), Thulium (Tm), Dysprosium (Dy), Ytterbium (Yb), Gadolinium (Gb), Europium (Eu), Terbium (Tb), Erbium (Er), Bismuth (Bi), or Indium (In). In some embodiments, the antibody is conjugated to a different isotope than the isotope detected in the biological sample. Representative isotopes that can be conjugated to the antibody include, but are not limited to, of ⁸⁹Y, ¹⁴¹Pr, ¹⁴²Nd, ¹⁴⁹Sm, ¹⁵²Sm, ¹⁷⁵Lu, ¹⁶⁵Ho, ¹⁶⁹Tm, ¹⁴⁶Nd, ¹⁶³Dy, ¹⁷¹Yb, ¹⁵⁸Gd, ¹⁷⁶Yb, ¹⁷³Yb, ¹⁵¹Eu, ¹⁵⁰Nd, ¹⁶²Dy, ¹⁴⁷Sm, ¹⁵⁹Tb, ¹⁵⁵Gd, ¹⁵⁴Sm, ¹⁶⁸Er, ¹⁶⁰Gd, ¹⁶¹Dy, ²⁰⁹Bi, ¹⁴⁴Nd, ¹⁷⁰Er, ¹⁴³Nd, ¹⁷⁴Yb, ¹¹³In, ¹⁶⁴Dy, ¹⁵³Eu, ¹⁵⁶Gd, ¹⁵⁷Er, and ¹⁷²Yb, or a combination or sub-combination thereof.

In some embodiments, the methods of the disclosure can be combined with methods for detecting one or more additional biological properties of the sample. For example, the methods can be combined with methods for detecting the phenotype or activation status of a cell isolated from a biological sample. For example, the methods can further detect the identity of an immune cell, or an antibody produced by a B cell. In some embodiments, the methods of the disclosure are combined with methods for analyzing the transcriptome or proteome of the biological sample. Methods for analyzing the transcriptome include detecting and/or sequencing RNA transcripts present in one or more cells of the biological sample, for example by hybridizing RNA sequences from the biological sample to a microarray or by sequencing the RNAs (or cDNAs prepared therefrom) present in the biological sample (e.g., RNAseq). Methods for analyzing the proteome include denaturing polyacrylamide gel electrophoresis (PAGE) or sodium dodecyl sulfate polyacrylamide gel electrophoresis (SDS-PAGE), two-dimensional gel electrophoresis, high-performance liquid chromatography (HPLC), capillary electrophoresis, affinity chromatography, mass spectrometry, and protein microarrays.

### Biological Samples

Biological samples that are suitable for the methods described herein include cells and tissues. The cell can be a single cell or isolated from a tissue sample. In some embodiments, the cell is a living cell. In some embodiments, the cell is a dead cell, for example, a cell that is fixed in a preservative such as formalin or obtained from a fixed tissue, or a cell that is cryopreserved or obtained from a cryopreserved tissue or dead cells stained with metal-tagged antibody. In some embodiments, the cell is obtained from a cadaver. The biological sample can be obtained from any organism, such as a human, an animal, a plant, or a microorganism, including a single-cell eukaryotic organism such as yeast, or a prokaryotic microorganism such as a bacterium.

In some embodiments, the biological sample is obtained from an organism exposed to heavy metals in the environment, such as heavy metal pollution. The organism can be exposed to heavy metals in water or air, for example in water polluted with heavy metals or heavy metals in air pollution from combustion of fossil fuels, or from wild-fire or forest-fire smoke. In some embodiments, the organism is a human. In some embodiments, the organism is a human child, for example, a human being less than one year old (e.g., 1 week, 2 weeks, 3 weeks, 1 month, 2 months, 3 months, 4 months, 5 months, 6 months, 7 months, 8 months, 9 months, 10 months, or 11 months old), or 1 year to 16 years old.

In some embodiments, the biological sample is labeled with one or more additional detectable labels, such as a fluorochrome, or a label that detects cell viability, a labeled metabolic pre-cursor molecule, and/or a barcode that is useful for identifying individual cells (see Kimmey, S. C. et al. “Parallel analysis of tri-molecular biosynthesis with cell identity and function in single cells.” Nature Communications 10 (2019): 1185.). In some embodiments, the biological sample is further labeled with one or more markers that detect chromatin modifications, one or more markers that detect total histones, one or more markers that detect post-translational modifications of histone proteins (“chromatin marks”) or one or more markers that are useful to determine the phenotype or identity of a cell. In some embodiments, the one or markers that that detect chromatin modifications, one or more marker that detects total histones, one or more markers that detect post-translational modifications of histone proteins (“chromatin marks”) or one or more markers that are useful to determine the phenotype or identity of a cell are conjugate to an antibody comprising a heavy metal mass tag. In some embodiments, the one or more chromatin markers are detected by Epigenetic landscape profiling using cytometry by Time-Of-Flight (EpiTOF) (see Cheung P., et al., Single-Cell Chromatin Modification Profiling Reveals Increased Epigenetic Variations with Aging, Cell 173, 1385-1397, 2018; see Cheung P., et al., SINGLE-CELL EPIGENETICS-CHROMATIN MODIFICATION ATLAS UNVEILED BY MASS CYTOMETRY. Clin Immunol. 2018 November: 196: 40-48)

In some embodiments, cells from the biological sample are sorted before being analyzed by mass cytometry. For example, cells can be sorted by fluorescence activated cell sorting (FACS) before being used in the methods described herein.

In some embodiments, the biological sample is obtained from a human. In some embodiments, the biological sample comprises one or more immune cells. In some embodiments, the biological sample comprises peripheral blood mononuclear cells (PBMC). In some embodiments, the biological sample comprises cryopreserved PBMC. In some embodiments, the immune cell is a dendritic cell, such as a plasmacytoid (pDC) or myeloid (mDC) dendritic cell. In some embodiments, the dendritic cell is a human dendritic cell and expresses one or more molecules or markers selected from BDCA-1, BDCA-2, BDCA-4, CD8, CD8 alpha, CD11b, CD11c, CD103, CD205, MHC class II, CD45RA, CD123, ILT-7, TLR7, and TLR9. In some embodiments, the dendritic cell does not express CD66, CD56, CD14, or CD16. In some embodiments, the dendritic cell is a CD66−/CD56−/CD14−/CD16− dendritic cell.

In some embodiments, the biological sample comprises leukocytes. In some embodiments, the leukocyte is selected from the group consisting of a B cell, a T cell, a myeloid cell, a granulocyte, a natural killer cell, or combinations thereof.

In some embodiments, the B cell is selected from the group consisting of transitional, naïve, activated, plasmablast, plasma cell, memory, B-1, B-2, and Regulatory (Breg) B cells, and combinations thereof. In some embodiments, the B cell expresses one or more molecules or markers selected from CD19, CD20, CD27, CD38, CD45, CD19, CD40, IgG, IgA, IgD, IgE, IgM, and Class II MHC.

In some embodiments, the T cell is selected from the group consisting of T cells expressing the alpha-beta chains of the T cell receptor (α/β-TCR), T cells expressing the gamma-delta chains of the T cell receptor (γ/δ-TCR), a helper T cell (e.g. a CD4+ T cell), a cytotoxic T cell (e.g. a CD8+ T cell), a memory T cell, a regulatory T cell (Treg), a CD8+ Treg, a natural killer T cell, and combinations thereof. In some embodiments, the helper T cell is a subtype selected from the group consisting of Th1, Th2, Th9, Th17, Th22, Tfh, and combinations thereof. In some embodiments, the cytotoxic T cell (Tc) is a subtype selected from the group consisting of Tc1, Tc2, Tc9, and Tc17. In some embodiments, the T cell expresses a molecule or marker selected from TCR-αβ, CD4, CD8, CD183 (CXCR3; Th1 and Tc1), CD194 (CCR4) and CD294 (CRTH2; both for Th2 and Te2), CD196 (CCR6) or CD161 (KLRB1; both for Th17 and Tc17), CCR10 (Th22), and CD25 (IL-2Ra) and CD152 (CTLA4; both for CD4+ and CD8+ Tregs).

In some embodiments, the myeloid cell is selected from neutrophils, monocytes, macrophages, myeloid dendritic cells (mDCs), and mast cells. In some embodiments, the granulocyte is a neutrophil, eosinophil, or basophil. In some embodiments, the monocyte expresses a molecule or marker selected from CD14 and CD16. In some embodiments, the monocyte does not express CD66 or CD56. In some embodiments, the monocyte is a CD66−/CD56−/CD14+ or CD16+ monocyte.

In some embodiments, the natural killer (NK) cell expresses a molecule or marker selected from CD133, CD34, CD33, CD117, CD244 (2B4), CD56 (CD56^{low} or CD56^{high}), CD94, and NKG2A. In some embodiments, the NK cell does not expresses the markers CD3. CD19 or CD66. In some embodiments, the NK cell is a CD56+/CD66− NK cell.

In some embodiments, the neutrophil expresses a molecule or marker selected from CD11b, CD16, CD32, CD44, CD55, and CD66. In some embodiments, the neutrophil does not express CD56. In some embodiments, the neutrophil is a CD56−/CD66+ neutrophil.

In some embodiments, the cell expresses a molecule or biomarker selected from CD45, CD45RO, CD3, CD19, CD56, CD66, CD66b, CD14, CD16, CD32, CD64, CD33, CD68, CD115, CD1c, CD141, CD123, FceR1a, CD11c, CD303, PD-11, CD36, CD163, CD206, CLEC4D, CD370, CD47, CD11b, CD54, CD62L, HLA-DR, CD86, CCR1, CCR2, CCR5, CCR7, and CX3CR1, or combinations or sub-combinations thereof.

In some embodiments, the cell is labeled with a detectable label. In some embodiments, the cell is contacted with one or more antibodies conjugated to a detectable label. In some embodiments, the one or more antibodies are conjugated to different detectable labels. In some embodiments, the one or more antibodies bind to one or more target molecules on the surface of the cell, and/or bind to one or more intracellular target molecules. In some embodiments, the one or more target molecules are different from each other. For example, the one or more antibodies can bind to one or more target molecules on an immune cell. In some embodiments, one or more antibodies bind to one or more of CD45, CD3, CD19, CD56, CD66, CD66b, CD14, CD16, CD32, CD64, CD33, CD68, CD115, CD1c, CD141, CD123, FceR1a, CD11c, CD303, PD-11, CD36, CD163, CD206, CLEC4D, CD370, CD47, CD11b, CD54, CD62L, HLA-DR, CD86, CCR1, CCR2, CCR5, CCR7, or CX3CR, or a combination or sub-combination thereof.

In some embodiments, the detectable label is an isotope of a heavy metal. In some embodiments, the detectable label is selected from an isotope of Yttrium (Y), Praseodymium (Pr), Neodymium (Nd), Samarium (Sm), Lutetium (Lu), Holmium (Ho), Thulium (Tm), Dysprosium (Dy), Ytterbium (Yb), Gadolinium (Gb), Europium (Eu), Terbium (Tb), Erbium (Er), Bismuth (Bi), or Indium (In). In some embodiments, the detectable label is selected from ⁸⁹Y, ¹¹Pr, ¹⁴²Nd, ¹⁹Sm, ¹⁵²Sm, ¹⁷⁵Lu, ¹⁶⁵Ho, ¹⁶⁹Tm, ¹⁴⁶Nd, ¹⁶³Dy, ¹⁷¹Yb, ¹⁵⁸Gd, ¹⁷⁶Yb, ¹⁷³Yb, ¹⁵¹Eu, ¹⁵⁰Nd, ¹⁶²Dy, ¹⁴⁷Sm, ¹⁵⁴Tb, ¹⁵⁵Gd, ¹⁵⁴Sm, ¹⁶⁸Er, ¹⁶⁰Gd, ¹⁶¹Dy, ²⁰⁹Bi, ¹⁴⁴Nd, ¹⁷⁰Er, ⁴³Nd, ¹⁷Yb, ¹¹³In, ¹⁶⁴Dy, ¹⁵³Eu, ¹⁵⁶Gd, ¹⁶⁷Er, and ¹⁷²Yb, or a combination or sub-combination thereof.

In some embodiments, the biological sample comprises a cell isolated from a tissue or organ within an organism, such as an animal, mammal, or human. In some embodiments, biological sample comprises a cell isolated from connective tissue, epithelial tissue, muscle tissue, or nervous tissue. In some embodiments, the biological sample comprises a tissue, or cell isolated therefrom, selected from the group consisting of bone, blood, lymph, adipose, cartilage, skin, mucous membranes, striated/skeletal muscle, smooth muscle, cardiac muscle, pancreas, heart, lungs, stomach, intestine, duodenum, liver, gall bladder, kidney, bladder, ovary, testes, prostate, uterus, placenta, fetal, eye, thyroid, parathyroid, salivary gland, breast or mammary gland, brain, spinal cord, and peripheral nervous system. In some embodiments, biological sample comprises placenta or fetal tissue, or a cell isolated therefrom. In some embodiments, the biological sample comprises a cell derived from endoderm, a cell derived from ectoderm or a cell derived from mesoderm. In some embodiments, the biological sample comprises a secretory cell of an exocrine gland, such as a salivary gland, Von Ebner's gland, mammary gland, lacrimal gland, ceruminous gland, eccrine sweat gland, apocrine sweat gland, gland of Moll, sebaceous gland, or Bowman's gland. In some embodiments, the biological sample comprises a hormone secreting cell of the pituitary gland, the hypothalamus, or adrenal gland. In some embodiments, the biological sample comprises a neuron selected from sensory neurons, autonomic neurons, a sense organ, a support cell such as a Schwann cell, a glial cell, an astrocyte, or an oligodendrocyte.

In some embodiments, the cell is a cardiac cell, a pulmonary cell, or a stem cell. In some embodiments, cell is a blood cell. In some embodiments, cell is a white blood cell (lymphocyte).

In some embodiments, the biological sample comprises a plant cell. In some embodiments, the biological sample comprises single celled organisms such as bacteria or yeast. In some embodiments, the biological sample comprises a cell from a parasite, a fungi, or any living organism comprising cells.

### Assays to Detect Heavy Metals

In some embodiments, the methods described herein comprise detecting heavy metals using mass cytometry, cytometry by time-of-flight (CyTOF®), inductively coupled plasma-mass spectrometry (ICP-MS), inductively coupled plasma time-of-flight mass spectrometry (ICP-TOF-MS), or epigenetic landscape profiling using cytometry by Time-Of-Flight (EpiTOF). Mass cytometry is a variation of flow cytometry in which antibodies are labeled with heavy metal ion tags rather than fluorochromes. The heavy metal ion tags can be detected by time-of-flight mass spectrometry. In some embodiments, the heavy metal ion tags are detected using CyTOF®. CyTOF® is a modification of mass cytometry that can be used to quantify labeled targets on the surface and interior of single cells. CyTOF allows the quantification of multiple cellular components simultaneously using an ICP-MS detector, which allows for the combination of many more antibody specificities in a single samples, without significant spillover between channels. In some embodiments, the heavy metals are detected using a Helios™ mass cytometer (Fluidigm, South San Francisco, CA).

Mass cytometry uses the same principal as ICP-MS but uses a cell-staining process with antibodies conjugated with heavy metals. Heavy metals, such as lanthides (14), are typically in low abundance in the environment and biological systems, and thus can be used to label cells with isotopes that are not present in a biological sample of interest. Thus, in some embodiments, the present disclosure provides a method for detecting HMs in cells from biological samples by detecting elements with atomic mass numbers between 75 and 209 which are not conjugated to antibodies (e.g. cadmium, antimony, mercury, and lead). Metal ion spectral overlaps between the heavy metals conjugated to antibodies used to label the cells and the heavy metals detected in the cell are not a concern given that they are minimal, ranging from 0-4% (15). Thus, in some embodiments, one (or a first) heavy metal is used to label the cell, and a different (or a second) heavy metal that was present in the biological sample is detected.

Briefly, mass cytometry comprises labeling cells with metal-conjugated probes, such as metal conjugated antibodies. The sample comprising the labeled cells is ionized with inductively coupled plasma, then the ions are separated by mass and quantified. In some embodiments, a suspension of the labeled cells are injected into a nebulizer, where they are aerosolized and directed through a spray chamber to a plasma torch. The cells are then vaporized, atomized, and ionized in the plasma. In some embodiments, a high pass optic removes the low-mass ions, resulting in an ion cloud that enters the TOF mass analyzer. The ions are separated based on their mass and are accelerated to a detector. The detector measures the quantity of each isotope for each individual cell in the sample; data is generated in an FCS format and analyzed, resulting in generation of a file that records the identity and amount of each probe for each cell. (See Helios, a CyTOF System. User Guide, SW Version 6.7, Fluidigm®).

Methods for performing mass cytometry are described, for example, in WO 2019/108554 A1 (U.S. Provisional Patent Application No. 62/591,701), U.S. Pat. No. 7,479,630 (Method and apparatus for flow cytometry linked with elemental analysis), U.S. Pat. No. 7,135,296 (Elemental analysis of tagged biologically active materials), and US published patent application 2008/0046194, which are incorporated by reference herein.

In another aspect, the disclosure provides methods for contacting cells with heavy metals in vitro, and detecting the expression of one or more biological markers associated with heavy metal exposure. In some embodiments, the biological marker is a marker of cell activation, such as CD69 or CD25. In some embodiments, the biological marker is a marker of a cell phenotype. In some embodiments, the biological marker comprises antibody production by a B cell. In some embodiments, changes in transcription rates or levels of one or more genes are detected after contacting the cell with a heavy metal. In some embodiments, changes in expression of one or more cytokines are detected after contacting the cell with a heavy metal. In some embodiments, the cell contacted with the heavy metal in vitro is an immune cell, such as a B cell. T cell, NK cell, or myeloid cell. In some embodiments, the heavy metal isotope(s) can be measured in the dead cell population, in addition to the live cell populations.

In another aspect, the disclosure provides methods for detecting the amount of heavy metals in a biological sample in vivo or ex vivo. In some embodiments, the methods comprise detecting heavy metals in a biological sample isolated from an animal model that was exposed to heavy metals. In some embodiments, the methods comprise detecting heavy metals in a biological sample comprising fetal tissue or placental tissue, or a cell isolated from fetal tissue or placental tissue.

The methods of the disclosure can be used to identify cells that contain heavy metals, and quantify levels for each metal isotope. Identification of cells that contain heavy metals and that express activation markers after culturing with standardized pollutants may improve assays involving lymphocyte stimulation or tissue analysis. The methods of the disclosure can increase our understanding of the impact of HM exposure on the immune system.

### EXAMPLES

### Example 1

This example describes a representative, non-limiting embodiment of the disclosure. People are at risk of heavy metal exposure due to its prevalence in the environment and from combustion sources, such as from wildfires and fossil fuels. This example describes a novel application of cytometry by time-of-flight (CyTOF) to simultaneously phenotype blood leukocytes and quantify the environmental heavy metals present in each cell, using samples from two cohorts: 1) firefighters with chronic exposure to smoke versus age and sex-matched controls not exposed chronically to smoke and 2) people with known chronic exposure to elevated air pollution in the San Joaquin Valley in California. Previously, typical biological heavy metals were detected with inductively coupled plasma-mass spectrometry. No method, however, existed to identify cell types at the single-cell level with intracellular or cell surface heavy metal content.

### Methods

Peripheral blood mononuclear cells (PBMCs) were isolated from cohorts using an IRB-approved protocol under Stanford University: 1) n=20 active duty firefighters recruited in 2021 from San Francisco training facility: 2) n=22 healthy controls that were age and sex-matched to the firefighters and not routinely exposed to wildfire smoke or elevated air pollution were recruited from Palo Alto, CA in 2019-2020; and 3) n=43 healthy and asthmatic 6-8-year-old children from Fresno, California, with recruitment and sample collection occurring within a 20 km radius of Fresno, CA, from 2015 to 2016. The PBMCs were isolated using the Ficoll-Paque assay and stored in liquid nitrogen until analysis. Samples were thawed in a 37° C. water bath and stained with a leukocyte-focused antibody panel (Biolegend, San Diego, CA, and Fluidigm Corporation, South San Francisco. CA), targeting dendritic cells and monocytes (Table 1). Data acquisition occurred using a Helios™ mass cytometer (Fluidigm Corporation, South San Francisco, CA). The presence of metal isotopes was measured in open stable isotope channels with no associated cell staining of metal-conjugated antibodies (Table 2) The open channels included bromide, cadmium, antimony, tantalum, tungsten, mercury, and lead isotopes. Data was analyzed using FlowJo™ software V10.7 (Becton, Dickinson & Company. Ashland, OR). GraphPad Prism (Version 8) was used for statistical analysis.

### Results

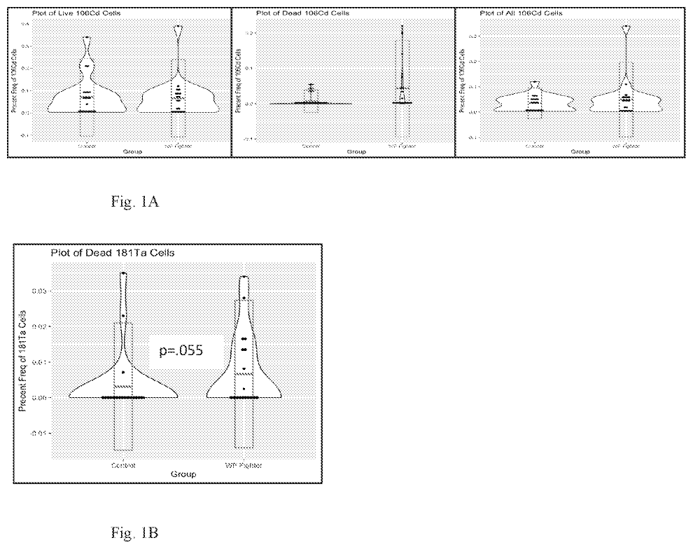

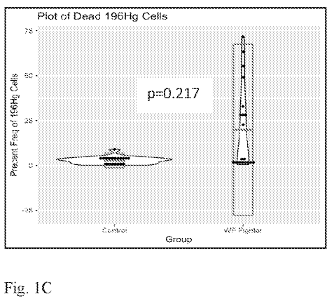

Manual gating was performed with FlowJo™ software. Live cells, dead cells and total cell populations for each heavy metal isotope were plotted. An example is shown in FIG. 1A for Cadmium isotope 106. Next, levels of heavy metal isotopes for the dead cell population in smoke-exposed firefighters (n=20) were compared to age and sex-matched controls (n=22) Examples are shown in FIG. 1B for Tantalum isotope 181 and FIG. 1C for Mercury isotope 196. Independent t-tests were performed comparing the groups with p values as noted.

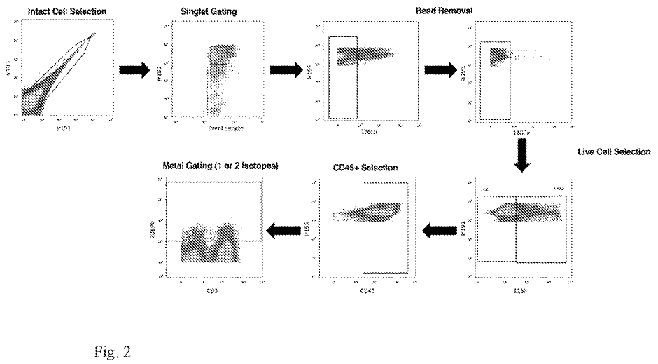

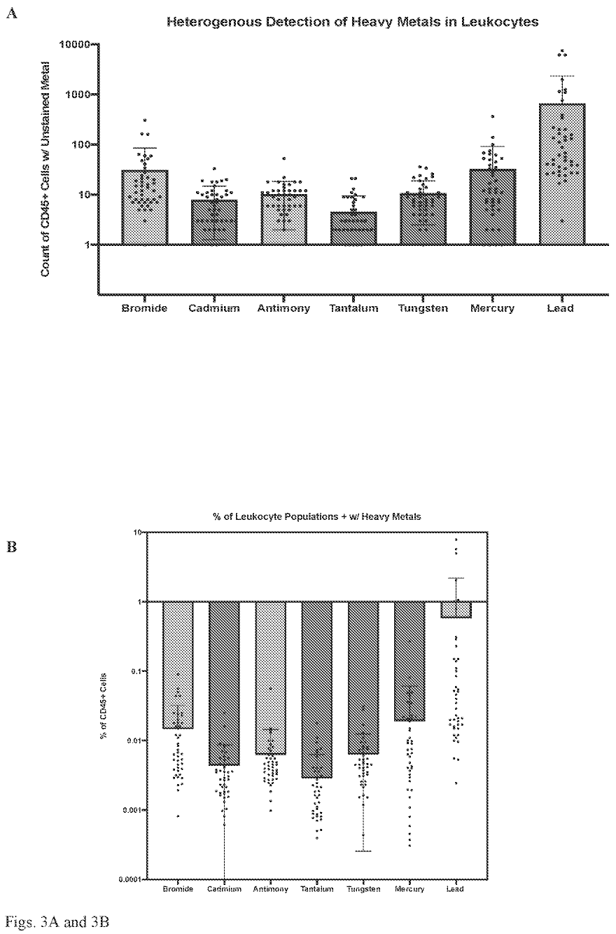

In a child population chronically exposed to air pollution (n=48), live cells were analyzed. Intact cells were first selected based on 193-Ir and 195-Ir staining and then doublets, which are multiple cells sticking together, were removed to avoid multiple cells being counted as a single cell. The 176Lu and 140Ce beads, which are known concentrations of the metal isotopes used to calibrate the CyTOF instrument, were also removed as standard practice. Subpopulations of CD45+ metal-positive leukocytes and dead cells were also measured (FIG. 2). Live cells were gated using 115 IN staining and CD45+ leukocytes were selected (CD45RO and/or CD45RA). Leukocytes were subsequently selected for environmental heavy metals for one or two isotopes at a time. Cell counts were successfully identified for bromide, cadmium, antimony, tantalum, tungsten, mercury, and lead (FIG. 3A). To standardize HM levels, counts were transformed into percent of total CD45+ cells for each sample (FIG. 3B). There was no overlap in the CyTOF readings between the heavy metal detection channels used for the antibodies vs those for bromide, cadmium, antimony, tantalum, tungsten, mercury and lead. Descriptive statistics were performed for both count and percentage, and large variability was observed between subjects (Tables 3A-3B).

Heavy metal isotope levels in the dead cell populations were compared in firefigthers versus controls (FIG. 1). Independent t-tests demonstrated a trend for higher heavy metal levels in the firefighters verus controls. This trend may not have been statitically significant due to the relatively small sample size.

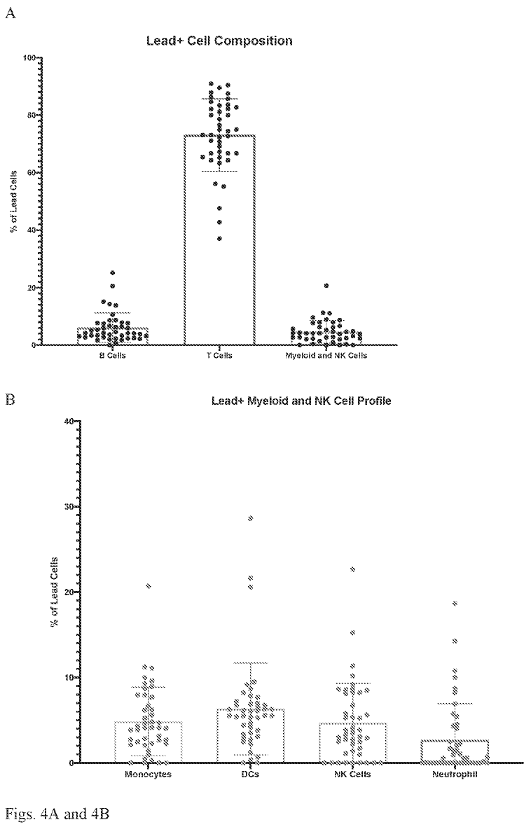

Lead levels were compared across leukocyte subtypes. The lead content for T cells, B cells, myeloid, and NK cells was measured (FIG. 4A). A Friedman test demonstrated a significantly larger distribution of lead in T cells compared to other cells (p<0.0001). Similar distributions of lead were observed in dendritic cells, monocytes, natural killer (NK) cells, and neutrophils (4B).

Correlational analyses was performed on the percentage of CD45 HM-containing cells with different subtypes of T cells: Th17 cells. T regulatory cells and Th1/Th2 ratio (Table 4). Using a false discovery rate (FDR) to provide corrected q-values, after correction there were significant correlations between the percentages of T regulatory cells and the level of cadmium (q<0.0022, r=0.56), and mercury (q<0.0022, r=0.64) (Tables 4A and 4B).

### Discussion

This example describes a novel method to measure HM exposure in blood cells while simultaneously phenotyping cells using traditional CyTOF procedures. By detecting isotopes in heavy metal channels for which no metal-associated cell staining was performed, the methods were able to detect bromide, cadmium, antimony, tantalum, tungsten, mercury, and lead within CD45+ leukocytes. HM levels were detected in all samples and the variation across subjects suggest distinct exposome profiles. In addition, testing and analysis with the exploratory data set demonstrates the ability to associate HM levels with cell profiles. Consistent with these findings, for example, Turley et al, reported that there was a moderate, yet significant increase in Tregs after low-level cadmium exposure in a rat model (16). As such, this adaption of mass cytometry may identify critical relationships between cell types and HMs.

In conclusion, toxic metals and their differences in exposed individuals can be measured. Toxic metal assessments via CyTof or other like methods will be helpful to determine exposures and potential toxicity at the acute and chronic level for human disease. This novel method of HM measurement and simultaneous cell phenotyping will increase our ability to understand the impact of HM exposure on the immune system.

### REFERENCES

1 Gr, T. A. J. (2011). Harmful Interactions of Non-Essential Heavy Metals with Cells of the Innate Immune System. Journal of Clinical Toxicology, s3. 10.4172/2161-0495.S3-005.
2. Gorr. M. W., Falvo, M. J., & Wold, L. E. (2017). Air Pollution and Other Environmental Modulators of Cardiac Function. Comprehensive Physiology, 7 (4), 1479-1495. doi.org/10.1002/cphy.c170017
3. Vouk V B, Piver W T, Metallic elements in fossil fuel combustion products: amounts and form of emissions and evaluation of carcinogenicity and mutagenicity. Environ Health Perspect. 1983 January: 47:201-25. doi: 10.1289/ehp.8347201. PMID: 6337825; PMCID: PMC1569408.
4. Al osman, M., Yang, F. & Massey, I. Y. Exposure routes and health effects of heavy metals on children. Biometals 32, 563-573 (2019), doi org.stanford.idm.oclc.org/10.1007/s10534-019-00193-5
5. Gundacker C, Hengstschläger M. The role of the placenta in fetal exposure to heavy metals. Wien Med Wochenschr. 2012 May; 162 (9-10): 201-6. doi: 10.1007/s10354-012-0074-3. PMID: 22717874.
6. Wai, K. M., Mar, O., Kosaka, S., Umemura, M., & Watanabe, C. (2017). Prenatal Heavy Metal Exposure and Adverse Birth Outcomes in Myanmar: A Birth-Cohort Study. International journal of environmental research and public health, 14 (11), 1339. doi.org/10.3390/ijerph14111339
7. Kippler, M., Tofail, F., Hamadani, J. D., Gardner, R. M., Grantham-McGregor, S. M., Bottai, M., & Vahter, M. (2012). Early-life cadmium exposure and child development in 5-year-old girls and boys: a cohort study in rural Bangladesh. Environmental health perspectives, 120 (10), 1462-1468. doi.org/10.1289/chp.1104431
8. Sherief L M, Abdelkhalek E R, Gharieb A F, Sherbiny H S, Usef D M, Almalky M A A, Kamal N M. Salama M A. Gohar W. Cadmium status among pediatric cancer patients in Egypt. Medicine (Baltimore). 2015 May; 94 (20): e740. doi: 10.1097/MD.0000000000000740. PMID: 25997039: PMCID: PMC4602885.
9. Mohamed, F., Zaky, E. A., El-Sayed. A. B., Elhossieny, R. M., Zahra, S. S., Salah Eldin, W., Youssef, W. Y., Khaled, R. A., & Youssef, A. M. (2015). Assessment of Hair Aluminum, Lead, and Mercury in a Sample of Autistic Egyptian Children: Environmental Risk Factors of Heavy Metals in Autism. Behavioural neurology, 2015, 545674. doi.org/10.1155/2015/545674
10. Kim, K. N., Kwon, H. J., & Hong, Y. C. (2016). Low-level lead exposure and autistic behaviors in school-age children. Neurotoxicology, 53, 193-200. doi.org/10.1016/j.neuro.2016.02.004
11. Appleton, A. A., Jackson. B. P., Karagas, M., & Marsit, C. J. (2017). Prenatal exposure to neurotoxic metals is associated with increased placental glucocorticoid receptor DNA methylation. Epigenetics, 12 (8), 607-615. doi.org/10.1080/15592294.2017.1320637
12, CDC. Laboratory Procedure Manual Analytes: Cadmium, Lead, Manganese, Mercury, and Selenium, Whole Blood. www.cdc.gov/nchs/data/nhanes/nhanes_11_12/PbCd_G_met_blood%20metals.pdf
13. Jones, D. R., Jarrett, J. M., Tevis, D. S., Franklin, M., Mullinix, N. J., Wallon, K. L., Derrick Quarles, C., Jr, Caldwell, K. L., & Jones, R. L. (2017). Analysis of whole human blood for Pb, Cd, Hg, Se, and Mn by ICP-DRC-MS for biomonitoring and acute exposures. Talanta, 162, 114-122. doi.org/10.1016/j.talanta.2016.09.060
14. Bjomson, Z. B., Nolan, G. P., & Fantl, W. J. (2013). Single-cell mass cytometry for analysis of immune system functional states. Current opinion in immunology. 25 (4), 484-494. doi.org/10.1016/j.coi.2013.07.004
15. Chevrier S, Crowell H L, Zanotelli V R T, Engler S, Robinson M D, Bodenmiller B. Compensation of Signal Spillover in Suspension and Imaging Mass Cytometry. Cell Syst. 2018 May 23; 6 (5): 612-620.e5. doi: 10.1016/j.cels.2018.02.010. Epub 2018 Mar. 28. PMID: 29605184: PMCID: PMC5981006.
16. Turley, A. E., Zagorski, J. W., Kennedy, R. C., Freebom, R. A., Bursley, J. K., Edwards, J. R., & Rockwell, C. E. (2019). Chronic low-level cadmium exposure in rats affects cytokine production by activated T cells. Toxicology research, 8(2), 227-237. doi.org/10.1039/c8tx00194d

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, may be referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the scope of this application.

While particular alternatives of the present disclosure have been disclosed, it is to be understood that various modifications and combinations are possible and are contemplated within the scope of the appended claims. There is no intention, therefore, of limitations to the exact abstract and disclosure herein presented.

TABLE 1
Added Antibodies.

| Antibody Marker | Metal Channel |
|---|---|
| CD45 | 89Y |
| CD3 | 141Pr |
| CD19 | 142Nd |
| CD56 (NCAM) | 149Sm |
| CD66b (CECAM8) | 152Sm |
| CD14, CD16 (FcgRIII), CD32 (FcgRII), | 175Lu, 165Ho, 169Tm, |
| CD64, CD33, CD68, CD115 (CSFR) | 146Nd, 163Dy, 171Yb, |
| | 158Gd |
| CD1c (BDCAI), CD141 (BDCA3), CD123 | 176Yb, 173Yb, 151Eu, |
| (IL3R), FceR1a, CD11c, CD303 (BDCA-2), | 150Nd, 162Dy, 147Sm, |
| PD-L1 | 159Tb |
| CD36, CD163, CD206 | 155Gd, 154Sm, 168Er |
| CLEC4D, CD370 (CLEC9A), CD47 | 160Gd, 161Dy, 209Bi |
| CD11b, CD54, CD62L | 144Nd, 170Er, 143Nd |
| HLA-DR, CD86 (B7.2) | 174Yb, 113In |
| CCR1, CCR2, CCR5, CCR7, CX3CR1 | 164Dy, 153Eu, 156Gd, |
| | 167Er, 172Yb |

TABLE 2
Open heavy metal channels monitored. There was no associated
cell staining of these metal-tagged antibodies.

| Metal on | |
|---|---|
| Periodic Table | Isotope Channels |
| Arsenic | 75As |
| Selenium | 78Se, 80Se |
| Bromine | 79Br, 81Br |
| Rhodium | 103Rh |
| Cadmium | 106Cd, 108Cd, 110Cd, 111Cd, 112Cd, 114Cd, 116Cd |
| Antimony | 121Sb, 123Sb |
| Barium | 138Ba |
| Tantalum | 180Ta, 181Ta |
| Tungsten | 182W, 184W, 186W |
| Osmium | 187Os, 1880s, 1890s |
| Platimun | 194Pt, 195Pt |
| Mercury | 196Hg, 198Hg, 199Hg, 200Hg, 201Hg, 202 Hg |
| Lead | 204Pb, 206Pb, 207Pb, 208Pb |

TABLE 3
Descriptive statistics (A) Count of metal positive cells, broken
down by element and (B) Percent of total CD45+ cells.

| | | | | | | |
|---|---|---|---|---|---|---|
| Bromide | Cadmium | Antimony | Tantalum | Tungsten | Mercury | Lead |

A

| | | | | | | | |
|---|---|---|---|---|---|---|---|
| Minimum | 1.00 | 1.00 | 1.00 | 0.00 | 1.00 | 0.00 | 3.00 |
| Maximum | 306.00 | 33.00 | 53.00 | 21.00 | 36.00 | 364.00 | 7481.00 |
| Range | 305.00 | 32.00 | 52.00 | 21.00 | 35.00 | 364.00 | 7478.00 |
| Mean | 31.30 | 7.96 | 10.27 | 4.57 | 10.70 | 32.30 | 663.20 |
| Std. Deviation | 54.48 | 6.70 | 8.29 | 4.88 | 8.18 | 59.42 | 1678.00 |
| Std. Error of | 8.21 | 1.01 | 1.25 | 0.74 | 1.23 | 8.96 | 252.90 |
| Mean | | | | | | | |

B

| | | | | | | | |
|---|---|---|---|---|---|---|---|
| Minimum | 0.001 | 0.001 | 0.001 | 0.000 | 0.000 | 0.000 | 0.002 |
| Maximum | 0.090 | 0.025 | 0.056 | 0.018 | 0.031 | 0.270 | 7.860 |
| Range | 0.089 | 0.024 | 0.055 | 0.018 | 0.031 | 0.270 | 7.858 |
| Mean | 0.015 | 0.004 | 0.006 | 0.003 | 0.006 | 0.019 | 0.585 |
| Std. Deviation | 0.018 | 0.004 | 0.008 | 0.004 | 0.006 | 0.042 | 1.613 |
| Std. Error of | 0.003 | 0.001 | 0.001 | 0.001 | 0.001 | 0.006 | 0.243 |
| Mean | | | | | | | |

TABLE 4
Correlation statistics. (A) Correlation analysis was performed between HMs and
Th1/Th2%, Th17%, and Treg %. Corrected q-values are presented. Significant corrected
q-values are bolded. (B) Pearson r, 95% confidence interval and R squared values
are presented for correlations with significant corrected q-values.
A

| | | | | | | Corrected |
|---|---|---|---|---|---|---|
| Heavy Metal % of | Th1/Th2% | Th1/Th2% | Th17% | Th17% | Treg % | Treg % |
| CD45+ Cells | P-Value | Q-Value | P-Value | Q-Value | P-Value | Q-Value |
| 78_80Selenium | 0.768 | 0.886 | 0.199 | 0.402 | 0.016 | 0.093 |
| 79Br_81Bromide | 0.279 | 0.517 | 0.969 | 0.913 | 0.837 | 0.886 |
| 112_116Cadmium | 0.483 | 0.715 | 0.027 | 0.121 | 0.000 | 0.002 |
| 121_123Antimony | 0.794 | 0.886 | 0.169 | 0.402 | 0.306 | 0.523 |
| 180Ta_181Tantalum | 0.194 | 0.402 | 0.986 | 0.913 | 0.557 | 0.727 |
| 182_184Tungsten | 0.130 | 0.402 | 0.116 | 0.402 | 0.798 | 0.886 |
| 196_198Mercury | 0.546 | 0.727 | 0.017 | 0.093 | 0.000 | 0.002 |
| 207_208Lead | 0.921 | 0.913 | 0.170 | 0.402 | 0.369 | 0.585 |

B

| | | 95% confidence | |
|---|---|---|---|
| | Pearson r | interval | R squared |
| 112_116Cadmium Treg % | 0.560 | 0.298 to 0.744 | 0.314 |
| 196_198Mercury Treg % | 0.642 | 0.409 to 0.796 | 0.412 |

### EXEMPLARY EMBODIMENTS

Exemplary embodiments provided in accordance with the presently disclosed subject matter include, but are not limited to, the claims and the following embodiments:

1. A method for detecting an amount of one or more heavy metals in a biological sample, the method comprising:
(a) contacting the biological sample with one or more antibodies conjugated to a heavy metal; and
(b) detecting both the amount of heavy metals conjugated to the one or more antibodies and the amount of the one or more heavy metals in the biological sample, wherein the one or more heavy metals in the biological sample are not conjugated to an antibody.

2. The method of embodiment 1, wherein the heavy metal conjugated to the one or more antibodies is different than the one or more heavy metals detected in the biological sample.

3. The method of embodiment 1 or 2, wherein the one or more heavy metals are detected by mass cytometry, cytometry by time-of-flight (CyTOF), inductively coupled plasma-mass spectrometry (ICP-MS), inductively coupled plasma time-of-flight mass spectrometry (ICP-TOF-MS) or epigenetic landscape profiling using cytometry by Time-Of-Flight (EpiTOF).

4. The method of any one of embodiments 1 to 3, wherein the one or more heavy metals comprise one or more isotopes in the range of 75-209 atomic mass units (AMU).

5. The method of any one of embodiments 1 to 4, wherein the one or more heavy metals are selected from the group consisting of arsenic, selenium, bromide, rhodium, cadmium, antimony, barium, tantalum, tungsten, osmium, platinum, mercury, lead, and combinations thereof.

6, The method of any one of embodiments 1 to 5, wherein the one or more isotopes are selected from the group consisting of 75As, 78Se, 80Sc, 79Br, 81Br, 103Rh, 106Cd, 108Cd, 110Cd, 111Cd, 112Cd, 114Cd, 116Cd, 121Sb, 123Sb, 138Ba, 180Ta, 181Ta, 182W, 184W, 186W, 187Os, 188Os, 189Os, 194 Pt, 195 Pt, 196Hg, 198Hg, 199Hg, 200Hg, 201Hg, 202Hg, 204Pb, 206Pb, 207Pb, 208Pb, and combinations thereof.

7. The method of any one of embodiments 1 to 6, wherein the one or more antibodies are conjugated to a heavy metal isotope in the range of 75-209 atomic mass units (AMU).

8. The method of any one of embodiments 1 to 7, wherein the antibody is conjugated to a heavy metal isotope selected from the group consisting of 89Y, 141Pr, 142Nd, 149Sm, 152Sm, 175Lu, 165Ho, 169Tm, 146Nd, 163Dy, 171Yb, 158Gd, 176Yb, 173Yb, 151Eu, 150Nd, 162Dy, 147Sm, 159Tb, 155Gd, 154Sm, 168Er, 160Gd, 161Dy, 209Bi, 144Nd, 170Er, 143Nd, 174Yb, 113 In, 164Dy, 153Eu, 156Gd, 167Er, 172Yb, and combinations thereof.

9. The method of any one of embodiments 1 to 8, wherein the antibody specifically binds to CD45, CD3, CD19, CD56, CD66, CD66b, CD14, CD16, CD32, CD64, CD33, CD68, CD115, CD1c, CD141, CD123, FceR1a, CD11c, CD303, PD-11, CD36, CD163, CD206, CLEC4D, CD370, CD47, CD11b, CD54, CD62L, HLA-DR, CD86, CCR1, CCR2, CCR5, CCR7, CX3CR1, or combinations thereof.

10. The method of any one of embodiments 1 to 9, wherein the biological sample comprises a cell or tissue.

11. The method of embodiment 10, wherein the cell is selected from the group consisting of an immune cell, a peripheral blood mononuclear cell (PBMC), a leukocyte, a dendritic cell, and combinations thereof.

12. The method of embodiment 11, wherein the leukocyte is selected from the group consisting of a B cell, a T cell, a myeloid cell, a granulocyte, a natural killer cell, and combinations thereof.

13. The method of embodiment 12, wherein the T cell is selected from the group consisting of a helper T cell, a cytotoxic T cell, a memory T cell, a regulatory T cell (Treg), a natural killer T cell, and combinations thereof.

14. The method of embodiment 13, wherein the helper T cell is a subtype selected from the group consisting of Th1, Th2, Th9, Th17, Tfh, and combinations thereof.

15. The method of any one of embodiments 10 to 14, wherein the cell is a single cell.

16. The method of any one of embodiments 10 to 15, wherein the cell is a living cell.

17. The method of any one of embodiments 10 to 14, wherein the cell is a dead cell, a cryopreserved cell, a fixed cell, or obtained from cadaveric tissue.

18. The method of any one of embodiments 10 to 17, wherein the cell or tissue is selected from an animal, a mammal, a human, a plant, a single cell eukaryotic organism, or a bacterium.

19. The method of any one of embodiments 1 to 18, wherein the heavy metal detected is on the cell surface.

20. The method of any one of embodiments 1 to 18, wherein the heavy metal detected is intracellular.

21. The method of any one of embodiments 1 to 20, wherein the biological sample is obtained from a subject exposed to heavy metals.

22. The method of any one of embodiments 1 to 21, wherein the biological sample is obtained from a subject exposed to air pollution.

What is claimed is:

1. A method for detecting an amount of one or more heavy metals in a biological sample, the method comprising:
(a) contacting the biological sample with one or more antibodies conjugated to a heavy metal; and
(b) detecting both the amount of heavy metals conjugated to the one or more antibodies and the amount of the one or more heavy metals in the biological sample, wherein the one or more heavy metals in the biological sample are not conjugated to an antibody.

2. The method of claim 1, wherein the heavy metal conjugated to the one or more antibodies is different than the one or more heavy metals detected in the biological sample.

3. The method of claim 1, wherein the one or more heavy metals are detected by mass cytometry, cytometry by time-of-flight (CyTOF), inductively coupled plasma-mass spectrometry (ICP-MS), inductively coupled plasma time-of-flight mass spectrometry (ICP-TOF-MS) or epigenetic landscape profiling using cytometry by Time-Of-Flight (EpiTOF).

4. The method of claim 1, wherein the one or more heavy metals comprise one or more isotopes in the range of 75-209 atomic mass units (AMU).

5. The method of claim 1, wherein the one or more heavy metals are selected from the group consisting of arsenic, selenium, bromide, rhodium, cadmium, antimony, barium, tantalum, tungsten, osmium, platinum, mercury, lead, and combinations thereof.

6. The method of claim 1, wherein the one or more isotopes are selected from the group consisting of ⁷⁵As, ⁷⁸Se, ⁸⁰Se, ⁷⁹Br, ⁸¹Br, ¹⁰³Rh, ¹⁰⁶Cd, ¹⁰⁸Cd, ¹¹⁰Cd, ¹¹¹Cd, ¹¹²Cd, ¹¹⁴Cd, ¹¹⁶Cd, ¹²¹Sb, ¹²³Sb, ¹³⁸Ba, ¹⁸⁰Ta, ¹⁸¹Ta, ¹⁸²W, ¹⁸⁴W, ¹⁸⁶W, ¹⁸⁷Os, ¹⁸⁸Os, ¹⁸⁹Os, ¹⁹⁴Pt, ¹⁹⁵Pt, ¹⁹⁶Hg, ¹⁹⁸Hg, ¹⁹⁹Hg, ²⁰⁰Hg, ²⁰¹Hg, ²⁰²Hg, ²⁰⁴Pb, ²⁰⁶Pb, ²⁰⁷Pb, ²⁰⁸Pb, and combinations thereof.

7. The method of claim 1, wherein the one or more antibodies are conjugated to a heavy metal isotope in the range of 75-209 atomic mass units (AMU).

8. The method of claim 1, wherein the antibody is conjugated to a heavy metal isotope selected from the group consisting of ⁸⁹Y, ¹⁴¹Pr, ¹⁴²Nd, ¹⁴⁹Sm, ¹⁵²Sm, ¹⁷⁵Lu, ¹⁶⁵Ho, ¹⁶⁹Tm, ¹⁴⁰Nd, ¹⁶³Dy, ¹⁷¹Yb, ¹⁵⁸Gd, ¹⁷⁶Yb, ¹⁷³Yb, ¹⁵¹Eu, ¹⁵⁰Nd, ¹⁶²Dy, ¹⁴⁷Sm, ¹⁵⁹Tb, ¹⁵⁵Gd, ¹⁵⁴Sm, ¹⁶⁸Er, ¹⁶⁰Gd, ¹⁶¹Dy, ³⁰⁰Bi, ¹⁴⁴Nd, ¹⁷⁰Er, ¹⁴³Nd, ¹⁷⁴Yb, ¹¹³In, ¹⁶⁴Dy, ¹⁵³Eu, ¹⁵⁶Gd, ¹⁶⁷Er, ¹⁷²Yb, and combinations thereof.

9. The method of claim 1, wherein the antibody specifically binds to CD45, CD3, CD19, CD56, CD66, CD66b, CD14, CD16, CD32, CD64, CD33, CD68, CD115, CD1c, CD141, CD123, FceR1a, CD11c, CD303, PD-II, CD36, CD163, CD206, CLEC4D, CD370, CD47, CD11b, CD54, CD62L, HLA-DR, CD86, CCR1, CCR2, CCR5, CCR7, CX3CR1, or combinations thereof.

10. The method of claim 1, wherein the biological sample comprises a cell or tissue.

11. The method of claim 10, wherein the cell is selected from the group consisting of an immune cell, a peripheral blood mononuclear cell (PBMC), a leukocyte, a dendritic cell, and combinations thereof.

12. The method of claim 11, wherein the leukocyte is selected from the group consisting of a B cell, a T cell, a myeloid cell, a granulocyte, a natural killer cell, and combinations thereof.

13. The method of claim 12, wherein the T cell is selected from the group consisting of a helper T cell, a cytotoxic T cell, a memory T cell, a regulatory T cell (Treg), a natural killer T cell, and combinations thereof.

14. The method of claim 13, wherein the helper T cell is a subtype selected from the group consisting of Th1, Th2, Th9, Th17, Tfh, and combinations thereof.

15. The method of claim 10, wherein the cell is a single cell.

16. The method of claim 10, wherein the cell is a living cell.

17. The method of claim 10, wherein the cell is a dead cell, a cryopreserved cell, a fixed cell, or obtained from cadaveric tissue.

18. The method of claim 10, wherein the cell or tissue is selected from an animal, a mammal, a human, a plant, a single cell eukaryotic organism, or a bacterium.

19. The method of claim 1, wherein the heavy metal detected is on the cell surface.

20. The method of claim 1, wherein the heavy metal detected is intracellular.

21. The method of claim 1, wherein the biological sample is obtained from a subject exposed to heavy metals.

22. The method of claim 1, wherein the biological sample is obtained from a subject exposed to air pollution.
